(19) United States
(12) Patent Application Publication
(10) Pub. No.: US 2004/0122396 A1
(43) Pub. Date: Jun. 24, 2004

(54) Apertured, film-coated nonwoven material

(76) Inventors: Jose E. Maldonado, Canton, GA (US); Steve Stopper, Duluth, GA (US)

Correspondence Address: PAULEY PETERSEN KINNE & ERICKSON, 2800 WEST HIGGINS ROAD, SUITE 365, HOFFMAN ESTATES, IL, 60195, US

(21) Appl. No.: 10/329,021

(22) Filed: Dec. 24, 2002

(51) Int. Cl. A61F 13/15; A61F 13/20

(52) U.S. Cl. 604/383

(57) ABSTRACT

An apertured, film-coated nonwoven fabric or material and a process for making the film-coated nonwoven fabric or material are provided. A nonwoven material layer is formed and extrusion coated with a polymer film to form a film-coated nonwoven material, the film-coated nonwoven material including a film layer having a thickness not greater than about 0.30 mils. A plurality of apertures may be formed in at least the film layer to form the apertured, film-coated nonwoven material. In alternative embodiments of this invention, a plurality of “peaks” or “cones” may be formed in the film layer having an aperture at a “valley” formed between adjacent peaks or cones.

### FIELD OF INVENTION

The present invention relates to a film-coated nonwoven fabric or material and a process for making the film-coated nonwoven fabric or material. The film-coated nonwoven fabric or material may include a plurality of apertures formed in at least the film layer.

### BACKGROUND OF THE INVENTION

Cover materials for personal care products should transmit liquid through from the wearer to the layers below the cover (or liner) material where the liquid may be absorbed or distributed to other areas. Liner materials preferably have low stain and low rewet surfaces in order to reduce the amount of liquid retained in the liner material itself. Apertured films are known in the art for use as liners because of their reduced staining and low rewetting. They do not, however, provide the softness and comfort of fibrous nonwoven liners. There remains, therefore, a need for a liner that provides the advantages of a film-based liner, while also being soft and comfortable for the wearer.

One objective of the present invention is to provide an absorbent material that may be used as a liner, which has low staining and rewetting and is soft and comfortable for the wearer. A further objective is for such a liner to also have greater strength than a film liner, and further, to enhance fluid handling functionality.

### SUMMARY OF THE INVENTION

The objects of the present invention are achieved by a cover material for an absorbent article including a thin film layer and a nonwoven material layer, wherein a film is extruded directly onto the nonwoven material layer to form a film-coated nonwoven material. The film-coated, nonwoven material may be made permeable by forming a plurality of apertures through at least the film layer.

The process of the present invention provides a nonwoven material layer. The nonwoven material layer may include a spunbond nonwoven web, a meltblown nonwoven web, a bonded carded web, an airlaid material, a coform material or laminates thereof, for example. Additional steps may be included in the process of the present invention, including, for example, crimping the fibers of the nonwoven material layer and/or through-air-bonding the nonwoven material layer prior to coating the nonwoven material layer with a thin polymer film layer. The polymer film layer suitably has a thickness of not greater than about 0.30 mils, and desirably not greater than about 0.28 mils. The extruded film layer may include any suitable polymer or polymers, such as polypropylene, low density polyethylene, linear low density polyethylene or a copolymer. In one embodiment of this invention, the film-coated nonwoven material can be passed through an aperturing apparatus or mechanism, such as hydro entangling, ultrasonic, and pattern calendar and anvil mechanisms, wherein a plurality of apertures is formed through at least the film layer. Alternatively, the aperturing apparatus may form a three-dimensional topography on an outer surface of the film layer having “peaks” and “valleys,” without forming apertures through the film layer.

The film-coated nonwoven material may provide improved high viscosity fluid intake with low rewet, cloth-like aesthetics and a clean, dry surface having good stain masking characteristics, good extensibility and recovery, good drapability and/or three-dimensional topography. The film-coated nonwoven material of the present invention may be used in a variety of product applications, for example personal care products such as diapers, training pants, and feminine care products, and health care products, such as window fenestration, drapes and surgical gowns. In alternative embodiments, the film-coated nonwoven material may be used as a mattress cover, a car cover, a bandage, a shoe insole lining or an acoustic material, for example.

### BRIEF DESCRIPTION OF THE DRAWINGS

These and other objects and features of this invention will be better understood from the following detailed description taken in conjunction with the drawings, wherein:

FIG. 1 is a schematic drawing of an apertured, film-coated nonwoven material, according to one embodiment of this invention;

FIG. 2 is a schematic drawing of an apparatus for forming a film-coated and apertured nonwoven material of the present invention; and

FIG. 3 is an exploded perspective view of an absorbent article having a film-coated nonwoven cover material, according to one embodiment of this invention.

### DESCRIPTION OF PREFERRED EMBODIMENTS DEFINITIONS

As used herein, the term “airlaying” is a well known process by which a fibrous nonwoven layer can be formed. In the airlaying process, bundles of small fibers having typical lengths ranging from about 6 to about 19 millimeters (mm) are separated and entrained in an air supply and then deposited onto a forming screen, usually with the assistance of a vacuum supply. The randomly deposited fibers then are bonded to one another using, for example, hot air or a spray adhesive.

As used herein, the term “biconstituent fibers” refers to fibers, which have been formed from at least two polymers extruded from the same extruder as a blend. Biconstituent fibers do not have the various polymer components arranged in relatively constantly positioned distinct zones across the cross-sectional area of the fiber and the various polymers are usually not continuous along the entire length of the fiber, instead usually forming fibrils or protofibrils which start and end at random. Biconstituent fibers are sometimes also referred to as multiconstituent fibers. Fibers of this general type are discussed in, for example, U.S. Pat. No. 5,108,827 to Gessner.

As used herein, the term “bonded carded web” refers to webs that are made from staple fibers which are sent through a combing or carding unit, which separates or breaks apart and aligns the staple fibers in the machine direction to form a generally machine direction-oriented fibrous nonwoven web. Such fibers are usually purchased in bales, which are placed in an opener/blender or picker that separates the fibers prior to the carding unit. Once the web is formed, it then is bonded by one or more of several known bonding methods. One such bonding method is powder bonding, wherein a powdered adhesive is distributed through the web and then activated, usually by heating the web and adhesive with hot air. Another suitable bonding method is pattern bonding, wherein heated calender rolls or ultrasonic bonding equipment are used to bond the fibers together, usually in a localized bond pattern, though the web can be bonded across its entire surface if so desired. Another suitable and well known bonding method, particularly when using bicomponent staple fibers, is through-air bonding.

As used herein, the term “co-extrusion” or “co-extruded” refers to films including two or more layers of thermoplastic material that are extruded simultaneously to form a single, integrated sheet of film without the need for a further attachment or lamination process to bond the layers together.

As used herein, the term “conjugate fibers” refers to fibers that have been formed from at least two polymer sources extruded from separate extruders but spun together to form one fiber. Conjugate fibers are also sometimes referred to as multicomponent or bicomponent fibers. The polymers are usually different from each other though conjugate fibers may be monocomponent fibers. The polymers are arranged in substantially constantly positioned distinct zones across the cross-section of the conjugate fibers and extend continuously along the length of the conjugate fibers. The configuration of such a conjugate fiber may be, for example, a sheath/core arrangement wherein one polymer is surrounded by another or may be a side by side arrangement, a pie arrangement or an “islands-in-the-sea” arrangement. Conjugate fibers are taught, for example, in U.S. Pat. No. 5,382,400 to Pike et al. For two component fibers, the polymers may be present in ratios of 75/25, 50/50, 25/75 or any other desired ratio. The fibers may also have shapes such as those described in U.S. Pat. No. 5,277,976 to Hogle et al., which describes fibers with unconventional shapes.

As used herein, the term “coform” means a process in which at least one meltblown diehead is arranged near a chute through which other materials are added to the web while it is forming. Such other materials may be pulp, superabsorbent particles, natural or synthetic staple fibers, for example. Coform processes are shown in commonly assigned U.S. Pat. No. 4,818,464 to Lau and U.S. Pat. No. 4,100,324 to Anderson et al. Webs produced by the coform process are generally referred to as coform materials.

As used herein and in the claims, the term “comprising” is inclusive or open-ended and does not exclude additional unrecited elements, compositional components, or method steps.

As used herein, the term “film” refers to a thermoplastic film made using a film extrusion process, such as a cast, blown or extrusion coating process.

As used herein, the term “hot air knife” or HAK means a process of prebonding or primarily bonding a newly produced microfiber web, particularly a spunbond microfiber web, in order to give it sufficient integrity, i.e. increase the stiffness of the web, for further processing, but does not mean the relatively strong bonding of secondary bonding processes like TAB, thermal bonding and ultrasonic bonding. A hot air knife is a device which focuses a stream of heated air at a very high flow rate, generally from about 1000 to about 10000 feet per minute (fpm) (305 to 3050 meters per minute), or more particularly from about 3000 to 5000 feet per minute (915 to 1525 m/min.) directed at the nonwoven web immediately after its formation. The air temperature is usually in the range of the melting point of at least one of the polymers used in the web, generally between about 200 and 550° F. (93 and 290° C.) for the thermoplastic polymers commonly used in spunbonding. The control of air temperature, velocity, pressure, volume and other factors helps avoid damage to the web while increasing its integrity. The HAK's focused stream of air is arranged and directed by at least one slot of about ⅛ to 1 inches (3 to 25 mm) in width, particularly about ⅜ inch (9.4 mm), serving as the exit for the heated air towards the web, with the slot running in a substantially cross-machine direction over substantially the entire width of the web. In other embodiments, there may be a plurality of slots arranged next to each other or separated by a slight gap. The at least one slot is usually, though not essentially, continuous, and may be comprised of, for example, closely spaced holes. The HAK has a plenum to distribute and contain the heated air prior to its exiting the slot. The plenum pressure of the HAK is usually between about 1.0 and 12.0 inches of water (2 to 22 mmHg), and the HAK is positioned between about 0.25 and 10 inches and more preferably 0.75 to 3.0 inches (19 to 76 mm) above the forming wire. In a particular embodiment the HAK plenum's cross sectional area for cross-directional flow (i.e. the plenum cross sectional area in the machine direction) is at least twice the total slot exit area. Since the forming wire on which the spunbond polymer web is formed generally moves at a high rate of speed, the time of exposure of any particular part of the web to the air discharged from the hot air knife is less than one tenth of a second and generally about one hundredth of a second in contrast with the through air bonding process which has a much larger dwell time. The HAK process has a great range of variability and controllability of many factors such as air temperature, velocity, pressure, volume, slot or hole arrangement and size, and the distance from the HAK plenum to the web. The HAK is further described in U.S. Pat. No. 5,707,468 to Arnold et al.

As used herein, the term “hydrophilic” describes films or fibers or the surfaces of films or fibers that are wetted by the aqueous liquids in contact with the film or fibers. The degree of wetting of the materials can, in turn, be described in terms of the contact angles and the surface tensions of the liquids and materials involved. Equipment and techniques suitable for measuring the wettability of particular film or fiber materials or blends of film or fiber materials can be provided by a Cahn SFA-222 Surface Force Analyzer System, or a substantially equivalent system. When measured with this system, films or fibers having contact angles less than 90° are designated “wettable” or hydrophilic, while films or fibers having contact angles equal to or greater than 90° are designated “nonwettable” or hydrophobic.

As used herein, the term “layer” when used in the singular can have the dual meaning of a single element or a plurality of elements.

As used herein, the term “liquid” means a nongaseous substance and/or material that flows and can assume the interior shape of a container into which it is poured or placed.

As used herein, the term “machine direction” or MD means the length of a fabric in the direction in which it is produced. The term “cross machine direction” or CD means the width of fabric, i.e. a direction generally perpendicular to the MD.

As used herein, the term “meltblown fibers” means fibers formed by extruding a molten thermoplastic material through a plurality of fine, usually circular, die capillaries as molten threads or filaments into converging high velocity, usually hot, gas (e.g. air) streams which attenuate the filaments of molten thermoplastic material to reduce their diameter, which may be to microfiber diameter. Thereafter, the meltblown fibers are carried by the high velocity gas stream and are deposited on a collecting surface to form a web of randomly disbursed meltblown fibers. Such a process is disclosed, for example, in U.S. Pat. No. 3,849,241 to Butin et al. Meltblown fibers are microfibers that may be continuous or discontinuous, are generally smaller than 10 microns (μm) in average diameter, and are generally tacky when deposited onto a collecting surface.

As used herein, the term “microfibers” means small diameter fibers having an average diameter not greater than about 75 microns, for example, having an average diameter of from about 0.5 microns to about 50 microns, or more particularly, microfibers may have an average diameter of from about 2 microns to about 40 microns. Another frequently used expression of fiber diameter is denier, which is defined as grams per 9000 meters of a fiber and may be calculated as fiber diameter in microns squared, multiplied by the density in grams/cc, multiplied by 0.00707. A lower denier indicates a finer fiber and a higher denier indicates a thicker or heavier fiber. For example, the diameter of a polypropylene fiber given as 15 microns may be converted to denier by squaring, multiplying the result by 0.89 g/cc and multiplying by 0.00707. Thus, a 15 micron polypropylene fiber has a denier of about 1.42 (15²×0.89×0.00707=1.415). Outside the United States the unit of measurement is more commonly the “tex”, which is defined as the grams per kilometer of fiber. Tex may be calculated as denier/9.

As used herein, the term “nonwoven material” or “nonwoven web” means a web having a structure of individual fibers or threads which are interlaid, but not in an identifiable manner as in a knitted fabric. Nonwoven materials or webs have been formed from many processes such as for example, meltblowing processes, spunbonding processes, and bonded carded web processes. The basis weight of nonwoven webs is usually expressed in ounces of material per square yard (osy) or grams per square meter (gsm) and the fiber diameters useful are usually expressed in microns (μm). (Note that to convert from osy to gsm, multiply osy by 33.91).

As used herein “pattern unbonded” or interchangeably “point unbonded” or “PUB,” means a fabric pattern having continuous bonded areas defining a plurality of discrete unbonded areas. The fibers or filaments within the discrete unbonded areas are dimensionally stabilized by the continuous bonded areas that encircle or surround each unbonded area, such that no support or backing layer of film or adhesive is required. The unbonded areas are specifically designed to afford spaces between fibers or filaments within the unbonded areas. PUB fabrics are disclosed in U.S. patent application Ser. No. 08/754,419, commonly assigned, the disclosure of which is incorporated herein by reference.

As used herein, the term “personal care product” means diapers, training pants, absorbent underpants, adult incontinence products, and feminine hygiene products.

As used herein, the term “polymer” includes, but is not limited to, homopolymers, copolymers, such as for example, block, graft, random and alternating copolymers, terpolymers, etc. and blends, and modifications thereof. Additionally, the term “polymer” also includes thermoplastic and thermoset polymers. Further, unless otherwise specifically limited, the term “polymer” shall include all possible geometrical configurations of the material. These configurations include, but are not limited to, isotactic, syndiotactic and atactic symmetries.

As used herein, the term “spunbond fiber” refers to small diameter fibers which are formed by extruding molten thermoplastic material as filaments from a plurality of fine capillaries of a spinnerette having a circular or other configuration, with the diameter of the extruded filaments then being rapidly reduced as by, for example, in U.S. Pat. No. 4,340,563 to Appel et al., and U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartmann, U.S. Pat. No. 3,502,538 to Petersen, and U.S. Pat. No. 3,542,615 to Dobo et al., each of which is incorporated herein in its entirety by reference. Spunbond fibers are quenched and generally not tacky when they are deposited onto a collecting surface. Spunbond fibers are generally continuous and often have average deniers larger than about 0.3, more particularly, between about 0.6 and 10.

As used herein, the term “through-air bonding” or “TAB” refers to a process of bonding a nonwoven bicomponent fiber web, wherein air, sufficiently hot to melt one of the polymers of which the web fibers are made, is forced through the web. The air velocity is between 100 and 500 feet per minute and the dwell time may be as long as 6 seconds. The melting and resolidification of the polymer provides the bonding. Through air bonding has relatively restricted variability and since through-air bonding requires the melting of at least one component to accomplish bonding, it is restricted to webs with two components like conjugate fibers or those, which include an adhesive. In the through-air bonder, air having a temperature above the melting temperature of one component and below the melting temperature of another component is directed from a surrounding hood, through the web, and into a perforated roller supporting the web. Alternatively, the through-air bonder may be a flat arrangement wherein the air is directed vertically downward onto the web. The operating conditions of the two configurations are similar, the primary difference being the geometry of the web during bonding. The hot air melts the lower melting polymer component and thereby forms bonds between the filaments to integrate the web.

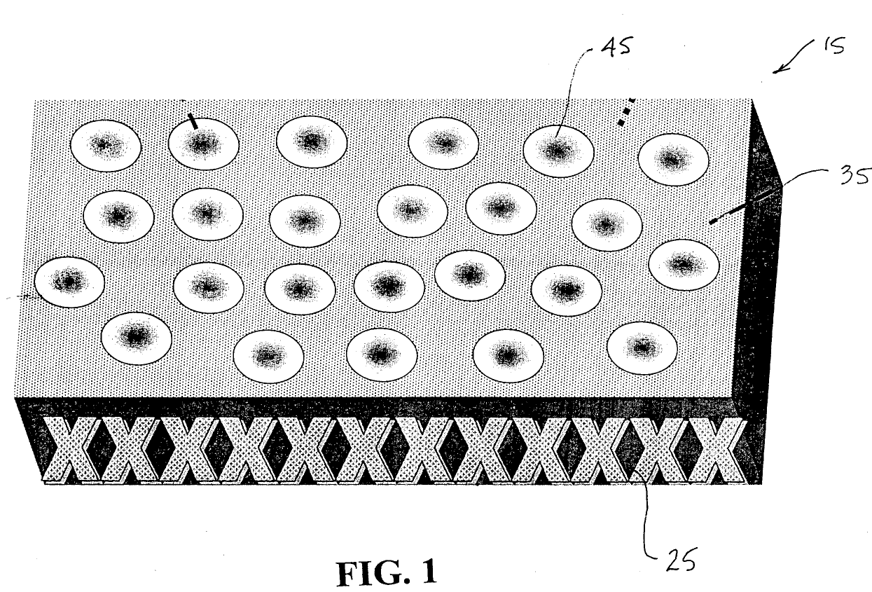

Referring to FIGS. 1-3, the present invention relates to an apertured, film-coated nonwoven material 15 and a process for producing the apertured, film-coated nonwoven material 15. The apertured, film-coated nonwoven material 15 of the present invention may be suitable for use in personal care products, such as diapers, training pants, adult incontinence articles, feminine care articles, other personal care garments, medical or health care garments, and other disposable articles and garments. For example, the film-coated nonwoven material 15 may be used as a diaper liner, a surge material, a spacer material, an outer cover or an extensible ear portion of an article. Further, the film-coated nonwoven material 15 may be used as a cover or liner material for feminine care products. In alternative embodiments, the film-coated nonwoven material 15 may be suitable for use as a car cover, a mattress cover, a bandage, a shoe insole lining or an acoustic material.

In one embodiment of this invention, the film-coated nonwoven material may include apertures through at least one material layer, but not necessarily completely through the thickness of the film-coated nonwoven material. For example, the apertures may be formed in the film layer to provide liquid communication between the film layer, the nonwoven material layer, and a subadjacent layer, such as a surge material layer. The apertures formed in the film layer may promote fluid intake, material flexibility, dimensional stability, and/or provide topography to an exterior surface of the apertured layer to reduce contact between the exterior surface of the material and a skin surface of the wearer to promote dryness, for example. The apertured, film-coated nonwoven material of the present invention is particularly useful in fabricating materials having extensibility, for example in the cross-machine direction, breathability, moisture vapor transmission, high viscosity fluid intake, liquid barrier properties and/or a clean surface appearance.

The apertured, film-coated nonwoven material 15 of the present invention includes a nonwoven material layer 25, for example a spunbond web, a meltblown web, a bonded carded web, an airlaid web, a coform material or laminates thereof. The nonwoven material layer 25 may be formed during an inline process or an offline process. The nonwoven material layer 25 may include any suitable fibers, such as monocomponent fibers, conjugate or multicomponent fibers, such as bicomponent fibers, biconsitutent fibers and combinations thereof. For example, the nonwoven material layer 25 may include a plurality of continuous side-by-side bicomponent spunbond fibers or a plurality of discontinuous staple fibers arranged as a bonded carded web.

A film layer 35 is applied to at least one surface of the nonwoven material layer 25 desirably using an extrusion process, wherein a thin layer of film material is extruded onto the nonwoven material layer 25. Desirably, the film layer 35 includes at least one polymer. In one embodiment of this invention, the film layer 35 may be a coextruded film layer including a polyolefin polymer layer and an adhesive-type polymer film layer, for example. Suitable adhesive-type polymers include, but are not limited to, heterophasic propylene-ethylene copolymers, propylene-ethylene random copolymers, ethylene vinyl acetate, ethylene-methyl acrylate, amorphous (Ziegler-Natta or single-site catalyzed) ethylene-alpha olefin copolymers having densities of about 0.89 grams/cm³ or less, amorphous poly-alpha olefin (APAO) polymers which can be random copolymers or terpolymers of ethylene, propylene and butene, other substantially amorphous or semi-crystalline propylene-ethylene polymers, EAA, EnBA, styrene-based elastomeric copolymers, anhydride modified versions thereof, available under the trade name BYNEL adhesive resins from E. I. Du Pont de Nemours Co., very low density LLDPE, and combinations of the foregoing.

In one suitable embodiment, the film layer 35 includes a mixture of a heterophasic propylene-ethylene polymer and an additional random propylene-ethylene copolymer. Heterophasic propylene-ethylene copolymers are available from Basell USA, Inc. (“Basell”) under the trade name ADFLEX®. Heterophasic polymers are reactor combinations of different polymer compositions produced, in sequence, in the same reactor and combined together. Heterophasic propylene-ethylene polymers are described in U.S. Pat. No. 5,300,365 to Ogale, the disclosure of which is incorporated herein by reference.

Additional suitable polymers for forming the film layer 35 using the extrusion process include, but are not limited to, polypropylene, low density polyethylene, liner low density polyethylene, copolymers, elastomeric polymers and combinations thereof.

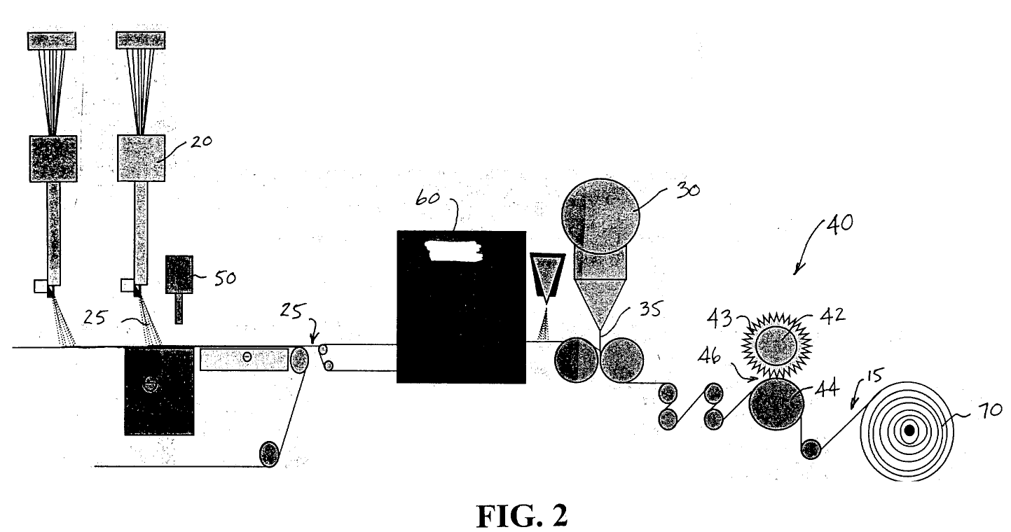

Referring to FIG. 2, a nonwoven material layer 25 is formed at a material forming apparatus 20 using a conventional process. For example, the nonwoven material 25 may comprise a nonwoven web or layer formed using a spunbond process, a bonded carded web process, a meltblown process or an airlaid process. In one embodiment of this invention, the nonwoven material includes a plurality of spunbond webs forming the nonwoven material layer 25 having a gradient fiber size structure. For example, during the nonwoven material layer forming process, a first spunbond machine may form a spunbond web having fibers of a first denier, and a second spunbond machine may form a second spunbond web having fibers of a second denier different from the first denier, to provide a gradient size structure across a thickness of the nonwoven material layer 25. In one embodiment of this invention, the nonwoven material layer 25 includes a spunbond nonwoven web including a plurality of continuous bicomponent fibers, such as side-by-side or sheath-core bicomponent fibers. Alternatively, the nonwoven material layer 25 may include any suitable nonwoven material known in the art, such as a bonded carded web material comprising a plurality of discontinuous staple fibers or an air laid nonwoven material, for example. Suitably, the nonwoven material layer 25 has a basis weight of about 0.4 osy to about 5.0 osy, desirably about 0.4 osy to about 3.0 osy, and in many cases about 0.4 osy to about 1.5 osy.

In one embodiment of this invention, after the nonwoven material layer 25 is formed, a film material is extruded onto the nonwoven material layer 25 using an extrusion process. For example, the nonwoven material layer 25 is conveyed or moved through an extrusion coating apparatus 30, as shown in FIG. 2, wherein the nonwoven material layer 25 is coated with a film layer 35. Desirably, the film material is extruded onto at least one of a first surface and an opposing second surface of the nonwoven material layer 25 to form a film-coated nonwoven material layer 25. Suitably, the film layer 35 has a thickness of not greater than about 0.30 mils, desirably not greater than about 0.28 mils, and in many cases not greater than about 0.20 mils. The relatively thin thickness of the film layer 25 provides flexibility and reduces manufacturing cost.

Suitable polymers for forming the nonwoven material layer 25 include, without limitation, certain polyolefins, polyamides, polyurethanes and polyesters. Exemplary polyolefins include one or more of polypropylene, polyethylene, ethylene copolymers, propylene copolymers, and butene copolymers. In one embodiment of this invention, the film layer 35 desirably includes a thermoplastic polymer, such as polypropylene, low density polyethylene, linear low density polyethylene, homopolymers, copolymers, elastomeric polymers and combinations thereof. It is apparent to those skilled in the art that other suitable polymers may be used to coat the nonwoven material 25, provided that the selection of polymer or polymers does not compromise the objects of the present invention. The film polymers suitably have a melt index of about 3 to about 30, desirably about 5 to about 20. Melt index is a measure of how easily a resin flows, and can be determined using ASTM Standard D1238, Condition 190/2.16.

In one embodiment of this invention the polymer film layer 35 can be made from any suitable elastomeric film-forming resins or blends containing the same. For example, materials suitable for use in preparing the elastomeric film layer include diblock, triblock, tetrablock, or other multi-block elastomeric copolymers such as olefinic copolymers, including styrene-isoprene-styrene, styrene-butadiene-styrene, styrene-ethylene/butylene-styrene, or styrene-ethylene/propylene-styrene, which may be obtained from Kraton Polymers, under the trade designation KRATON elastomeric resin or from SEPTON Company of America located in Pasadena, Texas under the trade designation SEPTON resins; polyurethanes, including those available from E. I. Du Pont de Nemours Co., under the trade name LYCRA polyurethane and urethane polymers available from Noveon, Inc., located in Cleveland, Ohio, under the trade name ESTANE urethane polymers; polyamides, including polyether block amides available from Atofina Chemical Company, under the trade name PEBAX polyether block amide; polyesters, such as those available from E. I. Du Pont de Nemours Co., under the trade name HYTREL thermoplastic polyester elastomer; and single-site or metallocene-catalyzed polyolefins having density less than about 0.89 grams/cubic centimeter, available from Dow Chemical Co. under the trade name AFFINITY.

As shown in FIG. 2, the film-coated nonwoven material layer 25 is conveyed or moved through an aperturing apparatus, such as a hot pin aperturing apparatus 40. Desirably, a plurality of apertures 45 are formed in at least one of the nonwoven material layer 25 and the film layer 35 as the film-coated nonwoven material 25 moves through the aperturing apparatus 40. The aperturing apparatus 40 comprises a pin roll 42 and a corresponding counter roll 44, which form a nip 46 therebetween, as shown in FIG. 2. Desirably, but not necessarily, the pin roll 42 and the counter roll 44 rotate at the same speed, i.e. a non-differential speed. The pin roll 42 comprises a plurality of pins or needles 43, which extend radially from a periphery of the pin roll 42 and are heated through induction. Each pin 43 may have any suitable length. For example, in one embodiment of this invention, each pin 43 has a length of about 0.5 mm to about 6.0 mm, desirably about 3.0 mm to about 5.0 mm. Further, each pin 43 is suitably heated to a temperature of about 20° C. to about 150° C., desirably about 80° C. to about 125° C., using conventional heating means known in the art. Each pin 43 may be pre-lubricated with a liquid, such as water, mineral oil or a surfactant for example. Pre-lubricating each pin 43 prior to contacting the film-coated nonwoven material 25 provides several benefits, particularly at high processing speeds. For example, at relatively high processing speeds, the apertures or holes produced typically become elongated or elliptical. Pre-lubricating the pins 43 promotes a more circular aperture or hole formation at higher speeds.

The pins 43 may have any suitable shape, depending on whether it is desirable to form the apertures 45 in the film-coated nonwoven material 15 as opposed to forming three-dimensional peaks or cones, for example. In one embodiment of this invention, the pins 43 have a generally conical cross sectional area along a height of the pin 43, and form a point at a radially extending end portion. Such pins 43 will form a plurality of apertures 45 through the film layer 35 and extend into the nonwoven material layer 25 as the material is passed through the nip 46. Alternatively, the pins 43 may form a blunt point forming a bullet-shaped pin or may have a flat surface at the radially extending end portion. Such bullet-shaped pins 43 form a plurality of peaks or cones and corresponding valleys between adjacent peaks or cones. Further, depending upon the pressure exerted on the material and the counter roll composition, i.e., the material used to form the outer surface of the counter roll 44, as the material passes through the nip 46, each pin 43 may form an aperture 45 at the corresponding formed valley, which may extend into the nonwoven material layer 25. Thus, three-dimensional peaks or cones may be formed on the film-coated surface of the material forming apertures, which extend into a thickness of the nonwoven material layer. Further, in one embodiment of this invention, the counter roll 44 may be heated to a temperature different than a temperature of the pin roll 42 to form a temperature gradient. Desirably, the counter roll 44 has a temperature gradient of at least about 5° C.

The counter roll 44 may be made of any suitable material, such as steel, a rubber or a silicone material, depending upon the desired aperturing. For example, the counter roll 44 may comprise a steel counter roll, wherein the counter roll 44 includes a plurality of apertures or bores, each countermatching and accepting at least a portion of a corresponding pin 43 of the pin roll 42, as the pin roll 42 and the counter roll 44 rotate and the pins 43 are pushed through the film-coated nonwoven material 15 to form apertures in the film-coated nonwoven material 15. Alternatively, the counter roll 44 may comprise a resilient rubber roll that provides a cushion as the pins 43 extend into the film-coated nonwoven material 15, preventing the pins from extending through the film-coated nonwoven material 15 to form apertures. As a result of using a resilient rubber counter roll 44, a plurality of three-dimensional peaks or cones may be formed in the film-coated nonwoven material 15 to provide a three-dimensional topography on the film-coated surface of the material 15.

In one embodiment of this invention, the film-coated nonwoven material layer 25 is passed through the nip 46 so that an outer surface of the film layer 25 faces the pin roll 42 to contact at least a portion of the pins 43 and an outer surface of the nonwoven material layer 25 faces or contacts the counter roll 44. As the film-coated nonwoven material layer 25 is passed between the nip 46, the apertures 45 are formed through at least a portion of the film layer 35 to form the apertured, film-coated nonwoven material 15. Desirably, the apertures 45 extend through at least the film layer 35. In one embodiment of this invention, the apertures 45 extend at least partially into the nonwoven material, and may extend through an entire thickness of the nonwoven material. A depth of the apertures 45 formed in the film-coated nonwoven material layer 25 can be controlled by varying a nip distance formed between the pin roll 42 and the counter roll 44 and/or a pressure applied to the material as the material passes through the nip 46.

For example, the nip distance may be set such that the pins 43 do not extend into the film layer 35 to form apertures 45, but only depress portions or areas of the film layer 35 to form a plurality of undulations or non-depressed areas on the exterior surface of the film layer 35. Alternatively, the pins 43 may form a plurality of apertures 45 through the film layer 35 that extend only into a portion but not through the nonwoven material layer 25. The undulations provide a topography to the exterior surface of the film layer 35. For example, the undulations may comprise cone-shaped landing areas that provide a cushion or soft feeling to the outer surface of the film layer 35. Such apertured, film-coated nonwoven material 15 may be used as a liner material in an absorbent article, for example, that contacts a skin surface of a wearer. The undulations formed on the exterior surface of the film layer 35 reduce the contact area between the outer surface of the film layer 35 and the skin surface to improve skin health and promote dryness. It can also be improved by adding a skin wellness additive or skin health benefit agent, as discussed below, to the surface or the film internally or topically.

In one embodiment of this invention, the process for forming the apertured, film-coated nonwoven material 15 may include additional steps before or after the film layer 35 is extruded onto the nonwoven material 15. For example, the process as shown in FIG. 2 may include a hot air knife 50. The nonwoven material layer 25 may be passed through the hot air knife 50 prior to passing the nonwoven material layer 25 through the extrusion coating apparatus 30. As the nonwoven material layer 25 is passed through the hot air knife 50, the fibers forming the nonwoven material layer 25 are primarily bonded to give the nonwoven material layer 25 sufficient integrity, i.e. increase the stiffness of the web. Such hot air knives and processes are known in the art. Additionally, the nonwoven material layer 25 may be passed through a through-air-bonding apparatus or mechanism 60. The fibers of the nonwoven material layer may also be crimped, using crimping apparatus and procedures known in the art, prior to passing the nonwoven material layer 25 through the extrusion coating apparatus 30. After the film layer 35 has been applied to the nonwoven material layer 25, the film layer 35 may be microembossed, using processes known in the art. Further, as shown in FIG. 2, the process of the present invention may also include a step of winding the apertured, film-coated nonwoven material 15 onto a storage roll 70 for subsequent use. Alternatively, the apertured, film-coated nonwoven material 15 may be conveyed or moved onto an inline manufacturing process, wherein a component for a personal care product, for example, is manufactured using the apertured, film-coated nonwoven material 15 formed by the process of the present invention.

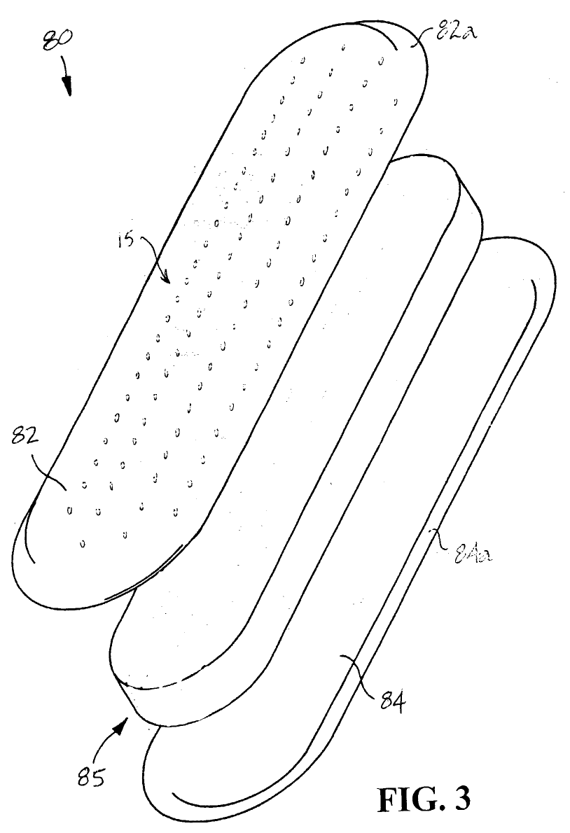

Referring to FIG. 3, there is illustrated an absorbent article, generally designated by the reference numeral 80, in accordance with one embodiment of the invention and which article is capable of absorbing body fluid. The absorbent article can be a diaper, training pant, sanitary napkin, panty liner, overnight pad, incontinence garment, underarm shield or other type of absorbent product capable of absorbing one or more bodily fluid such as urine, menses, blood, perspiration, excrement or the like. As will be appreciated, such an absorbent article will typically be disposable in the nature. While the absorbent article 80 will be described herein in terms of a feminine care product such as a sanitary napkin, it is to be understood that the broader practice of the invention is not necessarily so limited and that the invention can, if desired be practiced in or in association with other types or forms of absorbent articles such as identified above.

The absorbent article 80 comprises a generally liquid pervious liner or cover material 82 on the body-side surface of the article, a generally liquid impervious backsheet or baffle 84 on the opposing garment-facing side of the article and an absorbent core 85, disposed and enclosed therebetween.

It will be appreciated that absorbent articles such as feminine care products such as sanitary napkins may typically include additional standard or usual features such as relating to the positioning or placement of the article when in use. For example, certain sanitary napkin designs incorporate side flaps, sometimes referred to as “wings,” such as can be helpful in preventing fluid flow from the sides of the napkin. Another example of such a feature is the inclusion or presence of an adhesive at or about the garment facing region face of the backsheet. Such adhesive surface of the article can be covered by a release paper or the like, as is known in the art, prior to use such as when in a packaged state. As such features are standard or common, are well known to those skilled in that art and form no part of the broader invention, they will not be shown or described in great detail herein.

The liner 82 is generally designed to contact the body of the user and generally forms the contact surface of the absorbent article 80. In one embodiment of this invention, the liner 82 includes the apertured, film-coated nonwoven material 15 suitable for high viscosity fluid intake and stain masking. For example, the liner 82 may include the nonwoven material layer 25 for absorption and retention of bodily fluids and the film layer 35 extruded onto a surface of the nonwoven material layer 25.

In one embodiment of this invention, the nonwoven material layer 25 comprises a coform )material. A coform material suitable for use in this invention is available from the Kimberly-Clark Corporation located in Neenah, Wis. and is generally a nonwoven material made up of an air-formed matrix of thermoplastic polymer fibers and a multiplicity of individualized wood pulp fibers, and has a fabric-like finish. The thermoplastic fiber polymers generally have an average diameter of less than 10 microns with the individualized wood pulp fibers dispersed throughout the matrix and serving to space these microfibers from each other. The ratio of pulp fibers to microfibers is preferably in the range of about 10/90 to about 90/10, respectively. Thermoplastic polymers suitable for use in the coform material of this invention include polyolefins, for example, polyethylene, polypropylene, polybutylene and the like, polyamides, and polyesters. In accordance with a particularly preferred embodiment of this invention, the thermoplastic polymer used in the formation of the synthetic fibers of the coform material of this invention is polypropylene. The wood pulp fibers are interconnected by and held captive within the matrix of microfibers by mechanical entanglement of the microfibers with the wood pulp fibers, the mechanical entanglement and interconnection of the microfibers and wood pulp fibers alone forming a coherent integrated fiber structure. The coherent integrated fiber structure may be formed by the microfibers and wood pulp fibers without any adhesive, molecular or hydrogen bonds between the two different types of fibers. The wood pulp fibers are preferably distributed uniformly throughout the matrix of microfibers to provide a homogeneous material. The material is formed by initially forming a primary air stream containing the meltblown microfibers, forming a secondary air stream containing the wood pulp fibers, merging the primary and secondary streams under turbulent conditions to form an integrated air stream containing a thorough mixture of the microfibers and wood pulp fibers, and then directing the integrated air stream onto a forming surface to air form the fabric-like material. The microfibers are in a soft nascent condition at an elevated temperature when they are turbulently mixed with the wood pulp fibers in air. In one embodiment of this invention, the coform material is laminated with a secondary nonwoven fabric, for example, a spunbond liner.

In order to provide the coform material with improved fluid handling performance, the meltblown fibers can be sprayed with a surfactant treatment system comprising a compound selected from the group consisting of ethoxylated hydrogenated fatty oils, monosaccharides, monosaccharide derivatives, polysaccharides, polysaccharide derivatives, and combinations thereof. For example, the meltblown fibers can be sprayed with AHCOVEL Base N-62, a blend of hydrogenated ethoxylated castor oil and sorbitan monooleate, available from Hodgson Textile Chemicals, Mount Holly, N.C., U.S.A. Additionally, the secondary nonwoven fabric can also treated with a surfactant treatment system desirably comprising AHCOVEL Base N-62 or a blend of AHCOVEL Base N-62 and GLUCOPON 220 UP, a mixture of alkyl polyglycosides having 8-10 carbons in the alkyl chain. For treatment of the coform material, the surfactant treatment system has a relatively low solids content, typically about 3% AHCOVEL. For treatment of the secondary nonwoven fabric, the surfactant treatment system has a relatively high solids content, typically greater than about 10%.

At high solids content, AHCOVEL Base N-62 is very viscous and difficult to apply using conventional treating methods. Traditional viscosity modification additives or surfactant blends may reduce the viscosity of this treatment, but they adversely affect the durability of the treated fabric. Accordingly, in one embodiment of this invention, the surfactant treatment system applied to the meltblown fibers further comprises an alkyl polyglycoside that not only reduces the viscosity of the AHCOVEL Base N-62 treatment, but also maintains the desired fabric durability. For best results, the alkyl polyglycoside is one having 8 to 10 carbons in the alkyl chain and is provided in an amount of about 5% to about 50%, preferably about 6% to about 40%, based upon the total surfactant composition weight. In one embodiment of this invention, the allyl polyglycoside is GLUCOPON 220 UP, which comprises an octylpolyglycoside, available from Henkel Corporation, Ambler, Pa., U.S.A. Thus, the preferred surfactant treatment system for application to a coform material in accordance with this invention is a blend of AHCOVEL Base N-62 and GLUCOPON 220 UP (A/G) at ratios ranging from 1:1 to 20:1, respectively.

Numerous methods for hydrophilic treatment of nonwoven materials with surfactants having low solids content are known and are commonly used. However, due to the high solvent content, a drying step is required. It is known that the heat effects of the drying process negatively impact the mechanical properties of nonwoven materials following surfactant treatment. Thus, the use of a high-solids content treatment system, at least about 10% solids and advantageously at least about 20% solids, minimizes or alleviates the need for drying, thereby retaining the inherent tensile strength of the fabric. Other obvious advantages of a high-solids treatment system include lower cost for surfactant formulation, shipping and storage, conserved energy and lower treatment cost, and better treatment uniformity.

In one embodiment of this invention, the surfactant composition is applied to the meltblown and secondary nonwoven (spunbond) fibers at an add-on level ranging from about 0.1% to about 5% by weight. In accordance with one embodiment of this invention, the surfactant treatment system incorporates not only multiple surfactants for improved wettability with aqueous fluids, for example menstrual fluid, or for facilitating management of other bodily fluids (blood, urine, feces, etc.), but also include superabsorbents, bioactive compounds and macromolecules which may afford biofunctional attributes to the coform material of this invention, for example antibacterial activity, preservatives, anti-inflammatory, odor control, skin wellness, and the like.

Another material suitable for use as the nonwoven material layer 25 is the material known as PRISM available from Kimberly-Clark Corporation. A description of PRISM is taught in U.S. Pat. No. 5,336,552 to Strack et al. and the disclosure of that patent is incorporated by reference herein in its entirety. PRISM is generally the nonwoven fabric and comprises extruded multicomponent polymeric strands including first and second polymeric components arranged in substantially distinctive zones across the cross-section of the multicomponent strands and extending continuously along the length of the multicomponent strands. Preferably, the strands are continuous filaments that may be formed by spunbonding techniques. The second component of the strands constitutes at least a portion of the peripheral surface of the multicomponent strands continuously along the length of the multicomponent strands and includes a blend of a polyolefin and an ethylene alkyl acrylate copolymer. Bonds between the multicomponent strands may be formed by the application of heat. More specifically, the first polymeric component of the multicomponent strands is present in an amount of from about 20 to about 80 percent by weight of the strands and the second polymeric component is present in an amount from about 80 to about 20 percent by weight of the strands. Preferably, the first polymeric component of the multicomponent strands is present in an amount of from about 40 to about 60 percent by weight of the strands and the second polymeric component is present in an amount from about 60 to about 40 percent by weight of the strands.

The term “strand” as used herein refers to an elongated extrudate formed by passing a polymer through a forming orifice such a die. Strands include fibers, which are discontinuous strands having a definite length, and filaments, which are continuous strands of material. The nonwoven fabric of the present invention may be formed from staple multicomponent fibers. Such staple fibers may be carded and bonded to form the nonwoven fabric. Preferably, however, the nonwoven fabric of the present invention is made with continuous spunbond multicomponent filaments that are extruded, drawn and laid on a traveling forming surface.

The types of nonwoven materials that may be employed include powder-bonded-carded webs, infrared bonded carded webs, and through-air-bonded-carded webs. The infrared and through-air bonded carded webs can optionally include a mixture of different fibers, and the fiber lengths within a selected fabric web may be within the range of about 1.0 to 3.0 inch and an average bulk density of about 0.02 g/cc to about 0.12 g/cc.

In one embodiment of this invention, the nonwoven material layer 25 may be primarily bonded using a pattern-unbonded or “PUB” pattern to give the nonwoven material layer 25 a topography and minimize contact area with the wearer's skin surface. The PUB pattern may be formed on the nonwoven material layer 25 using a suitable process, wherein a nonwoven material layer 25 is passed through opposedly positioned first and second calender rolls that define a nip therebetween. Desirably, at least one of the rolls is heated and has a bonding pattern on its outermost surface including a continuous pattern of land areas defining a plurality of discrete openings, apertures or holes. Each of the openings in the at least one roll defined by the continuous land areas forms a discrete unbonded area in at least one surface of the nonwoven material layer 25 in which the fibers or filaments of the nonwoven material layer 25 are substantially or completely unbonded. Stated alternatively, the continuous pattern of land areas in the at least one roll forms a continuous pattern of bonded areas that define a plurality of discrete unbonded areas on at least one surface of the nonwoven material layer 25. Additionally, the nonwoven material layer 25 may be prebonded before passing the nonwoven material layer 25 through the nip formed by the calender rolls. Further, more than one nonwoven web may be provided to form a pattern-unbonded laminate.

In one embodiment of this invention, the nonwoven material layer 25 may comprise a first nonwoven material layer that is not wettable and a second nonwoven material layer that is wettable. Alternatively, the first nonwoven material layer may have a first wettability and the second nonwoven material layer may have a second wettability different from the first wettability. As a result, a surfactant gradient is formed across a thickness of the nonwoven material layer 25 including the first nonwoven material layer and the second nonwoven material layer.

At least one surface of the nonwoven material layer 25 is then coated with a thin film layer. For example, in one embodiment of this invention, a film layer 35 is applied to at least a surface of the nonwoven material layer 25 having a PUB pattern thereon, desirably using an extrusion process. Suitably, the film layer 35 has a thickness of not greater than about 0.30 mils, desirably not greater than about 0.28 mils, and in many cases not greater than about 0.20 mils. Desirably, the film layer 35 includes at least one polymer, such as a polyolefin. In one embodiment of this invention, the film layer 35 may be a co-extruded film layer including a polyolefin polymer layer and an adhesive-type polymer film layer, for example. Suitable polymers for forming the film layer 35 using the extrusion process include polypropylene, low density polyethylene, liner low density polyethylene, a copolymer, elastomeric polymers, and combinations thereof. The film layer 35 desirably provides a clean and dry appearance to the liner, in addition to masking stains.

In one embodiment of this invention, the film layer 35 may be treated with a surfactant, which may afford biofunctional attributes to the film layer 35 of this invention. For example, the surfactant composition can be applied to the film layer 35 at an add-on level ranging from about 0.1% to about 5% by weight. In accordance with one embodiment of this invention, the surfactant treatment system incorporates not only multiple surfactants for improved wettability with aqueous fluids, for example menstrual fluid, or for facilitating management of other bodily fluids (blood, urine, feces, etc.), but also include superabsorbents, bioactive compounds and macromolecules which may afford biofunctional attributes to the film layer of this invention, for example antibacterial activity, preservatives, anti-inflammatory, odor control, skin wellness, and the like. Suitable surfactants for use in the present invention to make the film layer 25 and/or the nonwoven material layer 25 wettable include, but are not limited to, a sodium neutralized anionic surfactant available under the trade name DOSS 70D from Manufactuer's Chemicals, L.P. located in Cleveland, Tenn., or a nonionic surfactant available under the trade name EMEREST® 2650 from Henkel Corporation located in Cincinnati, Ohio, or under the trade name SYNTHRAPOL® KB from Uniqema Corporation located in New Castle, Del., or under the trade name MASIL® SF-19 from BASF Corporation located in Mount Olive, N.J. The surfactant can be applied internally or topically to the film layer 35 and/or the nonwoven material layer 25.

In one embodiment of this invention, the film layer 35 may be treated with a lipophilic skin health benefit agent. As used herein, the phrase “lipophilic skin health benefit agent” is defined as any substance that has a higher affinity for oil over water and provides a skin health benefit by directly interacting with the skin. Suitable examples of such benefits include, but are not limited to, enhancing skin barrier function, enhancing moisturization and nourishing the skin.

The lipophilic skin health benefit agents may include stearic acid, isoparrafin, petrolatum, and a combination thereof. The lipophilic skin health benefit agent can also be selected from fatty acids, fatty acid esters, fatty alcohols, triglycerides, phopholipids, mineral oils, essential oils, sterols, sterol esters, emollients, waxes, and a combination thereof. In some embodiments, the lipophilic skin health benefit agent has an average hydrocarbon chain with length greater than eight carbons (C-8). An example of a lipophilic skin health benefit lotion composition is commercially available as Vaseline® Intensive Care Lotion (Cheesborough-Ponds, Inc.).

Humectants may also be included in the composition to provide an enhanced barrier and/or skin moisturization benefit. Humectants are typically cosmetic ingredients used to increase the water content of the top layers of the skin. This group of materials includes primarily hydroscopic ingredients. As used herein, suitable humectants include, but are not limited to, the following materials Acetamide MEA, Aloe Vera Gel, Arginine PCA, Chitosan PCA, Copper PCA, Corn Glycerides, Dimethyl Imidazolidinone, Fructose, Glucamine, Glucose, Glucose Glutamate, Glucuronic Acid, Glutamic Acid, Glycereth-7, Glycereth-12, Glycereth-20, Glycereth-26, Glycerin, Honey, Hydrogenated Honey, Hydrogenated Starch Hydrolysate, Hydrolyzed Corn Starch, Lactamide MEA, Lactic Acid, Lactose Lysine PCA, Mannitol, Methyl Gluceth-10, Methyl Gluceth-20, PCA, PEG-2 Lactamide, PEG-10 Propylene Glycol, Polyamino Sugar Condensate, Potassium PCA, Propylene Glycol, Propylene Glycol Citrate, Saccharide Hydrolysate, Saccharide Isomerate, Sodium Aspartate, Sodium Lactate, Sodium PCA, Sorbitol, TEA-Lactate, TEA-PCA, Urea, Xylitol, and the like, as well as mixtures thereof.

The composition may also include emulsifying surfactants. The surfactants include, but are not limited to, sorbitan monoleate, sorbitan seequioleate, sorbitan trioleate, glyceryl stearate, sorbitan stearate, sorbitan tristearate, and the like, as well as mixtures thereof.

The composition may also include viscosity enhancers. As used herein, suitable viscosity enhancers include, but are not limited to, the following materials: the group consisting of polyolefin resins, polyolefin polymers, ethylene/vinyl acetate copolymers, polyethylene, and the like, as well as mixtures thereof. Lipophilic skin health benefit agent lotion compositions can include humectants, surfactants, and viscosity enhancers present in an amount ranging from about 0.1% to about 10.0% of the total weight of the lipophilic skin health benefit agent composition.

It will be apparent to those skilled in the art that additional agents may be desirable for inclusion in the present composition. Examples include, but are not limited to, acceptable carriers, anti-inflammatories, antimicrobials, anti-puretics, skin protectants, buffering agents, α-hydroxy acids, microbial or algal extracts and/or fractions thereof, enzyme inhibitors, antihistamines, antioxidants, analgesics, antioxidants, astringents, fragrances, dyes, natural and/or synthetic vitamin analogs, sunscreens, deodorants, and combinations thereof.

The film layer 35 may also include at least one filler and/or dye, such as a pigment. Suitable filler materials include, but are not limited to, a suitable particulate inorganic filler, such as calcium carbonate, clays, silica, alumina, barium sulfate, sodium carbonate, talc, magnesium sulfate, titanium dioxide, zeolites, aluminum sulfate, diatomaceous earth, magnesium sulfate, magnesium carbonate, barium carbonate, kaolin, mica, carbon, calcium oxide, magnesium oxide, aluminum hydroxide and combinations of these particles.

In one embodiment of the invention, the film layer 35 may be colored. The color may be applied to the film layer 35 in the form of a dye that remains with the film layer 35 and does not transfer to other surfaces. The dye may mix with polymers in the film layer 35 to prevent the dye from transferring to other surfaces. The concentration of color in the polymer is dependent on the dye, but typically ranges from 0.1% to about 85%.

Examples of suitable types of dyes include acid, azo, basic, direct, disperse, solvent, mordant, reactive, pigment, sulfur, vat, organic, and natural dyes, and combinations of any of these. The most common types of natural dyes are acid or anionic dyes, such as indigo or Tyrian purple. A dye used in the particles may be virtually any color, and in particular, may be either fluorescent or non-fluorescent. Examples of suitable FDC dyes include tartrazine (Yellow #5—lemon yellow), sunset yellow (Yellow #6—orange), erythrosine (Red #3—cherry red), allura red AC (Red #40—orange red), brilliant blue FCF (Blue #1—bright blue), indigotine (Blue #2—royal blue), and fast green FCF (Green #3—sea green). Examples of commercially available fluorescent pigments are found in the line of Fluorescent Pigments 800P Series, available from Chempon Dyes P Ltd of Chicago, Ill. These fluorescent pigments are thermoset fluorescent pigments having a resistance to strong solvents. These pigments are available in a wide range of colors. Examples of commercially available fiber reactive dyes include CIBACRON F and REACTONE, both available from CIBA-Geigy Ltd. of Basle, Switzerland; PROCION MX and PROCION H, both available from ICI of Great Britain; LEVAFIX, available from Bayer Aktiengesellschaft of Germany; DRIMARENE, available from Sandoz Inc. of New York; CAVALITE, available from E. I. Du Pont de Nemours Co. of Wilmington, Del.; PRIMAZIN, available from BASF of Germany; and REMAZOL, available from Hoechst Aktiengesellschaft of Germany.

As discussed above in reference to FIG. 2, a plurality of apertures 45 is desirably formed through the film-coated nonwoven material 25 as the film-coated nonwoven material 25 moves through the aperturing apparatus 40. The apertures 45 formed in the film-coated nonwoven material layer 25 provide for high viscosity fluid intake as well as breathability.

The backsheet or baffle 84 is generally liquid impermeable and is designed to face the inner face, e.g., the crotch portion, of an undergarment (not shown). The backsheet 84 may desirably be designed to permit the passage of air or vapor out of the absorbent article 80 while preventing or blocking the passage of fluids therethrough. As will be appreciated, the backsheet 84 can be made of any suitable material capable of providing or having the above-identified properties or characteristics. For example, suitable materials may include a microembossed polymeric film such as of polyethylene or polypropylene.

As will be appreciated, the liner 82 and the backsheet 84 can be placed coextensive, in face-to-face contact around or about the absorbent core 85. Further, the topsheet 82 has a periphery 82a and the backsheet 84 has a periphery 84a which are desirably joined or sealed together by use of an adhesive, by heat sealing ultrasonics or other suitably selected techniques such as are known to those skilled in the art.

The absorbent core 85 for use in the practice of the invention can be fabricated or formed of various suitable absorbent materials such as are known in the art. For example, the absorbent core 85 can be fabricated or formed of various hydrophilic types of natural or synthetic fibers including cellulose fibers, surfactant-treated meltblown fibers, wood pulp fibers, regenerated cellulose, cotton fibers or a blend of other fibers. Absorbent core materials of construction can also include a coform material, as discussed above in reference to the liner 82.

The absorbent core 85 may suitably be composed of a matrix of hydrophilic fibers, such as a web of cellulosic fluff, mixed with particles of a high-absorbency material commonly known as superabsorbent material. In one embodiment of this invention, the absorbent core 85 includes a matrix of cellulosic fluff such as wood pulp fluff and superabsorbent hydrogel-forming particles. The wood pulp fluff may be exchanged with synthetic, polymeric, meltblown fibers, or with a combination of meltblown fibers and natural fibers. The superabsorbent particles may be substantially homogeneously mixed with the hydrophilic fibers or may be nonuniformly mixed. The fluff and superabsorbent particles may also be selectively placed into desired zones of the absorbent core 85 to better contain and absorb body exudates. The concentration of the superabsorbent particles may also vary through the thickness of the absorbent core 85. Alternatively, the absorbent core 85 may comprise a laminate of fibrous webs and superabsorbent material or other suitable means of maintaining a superabsorbent material in a localized area.

Suitable high-absorbency materials for the absorbent core 85 include, but are not limited to, natural, synthetic, and modified natural polymers and materials. The high-absorbency materials can be inorganic materials, such as silica gels, or organic compounds, such as crosslinked polymers. The term “crosslinked” refers to any means for effectively rendering normally water-soluble materials substantially water insoluble but swellable. Such means can include, for example, physical entanglement, crystalline domains, covalent bonds, ionic complexes and associations, hydrophilic associations such as hydrogen bonding, and hydrophobic associations or Van der Waals forces.

Examples of suitable synthetic, polymeric, high-absorbency materials include, but are not limited to, the alkali metal and ammonium salts of poly(acrylic acid) and poly(methacrylic acid), poly(acrylamides), poly(vinyl ethers), maleic anhydride copolymers with vinyl ethers and alpha-olefins, poly(vinyl pyrolidone), poly(vinyl morpholinone), poly(vinyl alcohol), and mixtures and copolymers thereof. Further polymers suitable for use in the absorbent core 85 include, but are not limited to, natural and modified natural polymers, such as hydrolyzed acrylonitrile-grafted starch, acrylic acid grafted starch, methyl cellulose, carboxymethyl cellulose, hydroxypropyl cellulose, and the natural gums, such as alginates, xanthan gum, locust bean gum, and similar compounds. Mixtures of natural and wholly or partially synthetic absorbent polymers can also be useful in the present invention. Such high-absorbency materials are well known to those skilled in the art and are widely commercially available. Examples of superabsorbent polymers suitable for use in the present invention are SANWET IM 3900 polymer available from Hoechst Celanese located in Portsmouth, Va. and DOW DRYTECH 2035LD polymer available from Dow Chemical Co. located in Midland, Mich.

The high absorbency material may be in any of a wide variety of geometric forms. Generally, it is desired that the high absorbency material be in the form of discrete particles. However, the high absorbency material may also be in the form of fibers, flakes, rods, spheres, needles, or the like. Generally, the high absorbency material is present in the absorbent core 85 in an amount of about 5 weight percent to about 90 weight percent, based on a total weight of the absorbent core 85.

In certain embodiments, the use of absorbent materials in the nature of surge materials may be desired. Various woven fabrics and nonwoven webs can be used to construct surge materials. For example, a surge material may be a nonwoven fabric layer composed of a meltblown or spunbond web of polyolefin filaments. Such nonwoven fabric layers may include conjugate, biconstituent and homopolymer fibers of staple or other lengths and mixtures of such fibers with other types of fibers. The surge material also can be a bonded carded web or an airlaid web composed of natural and/or synthetic fibers. The bonded carded web may, for example, be a powder bonded carded web, an infrared bonded carded web, or a through-air bonded carded web. The bonded carded webs can optionally include a mixture or blend of different fibers, and the fiber lengths within a selected web may range from about 3 mm to about 60 mm.

Examples of particular surge materials may be found in U.S. Pat. No. 5,490,846 to Ellis et al. and in U.S. Pat. No. 5,364,382 to Latimer. Surge materials may be composed of a substantially hydrophobic material, and the hydrophobic material may optionally be treated with a surfactant or otherwise processed to impart a desired level of wettability and hydrophilicity.

Other suitable absorbent materials for use in the practice of the invention can include materials commonly referred to as superabsorbents. Superabsorbents can be in various forms including particulate and fibrous forms. Known superabsorbent materials include AFA-1 30-53C by Dow Chemical, and W77553 and FAV880A that are commercially available from the Stockhausen Company of Greensboro, N.C. Stockhausen's W77553 is a bulk polymerized polyacrylate with a hydrophobic surface treatment. Stockhausen's FAV880A is a highly crosslinked surface superabsorbent. AFA 130-53C is a 850 to 1400 micron suspension polymerized polyacrylate material available from The Dow Chemical Company of Midland, Mich.

Hydrocolloidal materials, commonly referred to as superabsorbents, can be in the form of a hydrogel-forming polymer composition which is water-insoluble, slightly cross-linked, and partially neutralized. It can be prepared from an unsaturated polymerizable, acid group-containing monomers and cross-linked agents. Such superabsorbents are taught in U.S. Pat. No. 4,798,603 to Meyers et al., U.S. Reissue Pat. No. 32,649 to Brandt et al. and U.S. Pat. No. 4,467,012 to Pedersen et al., as well as in published European Patent Application 0,339,461 to Kellenberger. The disclosures of these patents and the European Patent Application are incorporated by reference herein in their entirety.

Suitable absorbent materials for use in the practice of the invention may also take the form of absorbent foams such as open cell polyurethane foam, such as disclosed in U.S. Pat. No. 5,853,402 to Faulks et al., the disclosure of which patent is incorporated herein its entirety. Further, starch foams such as disclosed in U.S. Pat. No. 5,506,277 to Griesbach III, the disclosure of which patent is incorporated herein its entirety, may also be used.

The invention may also utilize, as suitable absorbent materials, corrugated nonwoven fabrics such as the high bulk corrugated nonwoven fabric disclosed in U.S. Pat. No. 3,668,054 to Stumpf, the disclosure of which patent is incorporated herein its entirety. As disclosed therein, such fabric generally comprises a corrugated web of initially aligned textile fibers implanted in a continuous thin film of a thermoplastic adhesive having an essentially constant thickness. The resulting web-adhesive material is then corrugated to provide the multitude of furrows and grooves, which are irregularly connected near their roots and along their respective sides.

In the practice of the invention, adjacent absorbent members can, if desired, be loosely plied or, if desired, bonded to one another such as via the use of adhesives, thermal or ultrasonic techniques, threading or sewing techniques or other suitable joining technique such as known in the art.

While in the foregoing specification this invention has been described in relation to certain preferred embodiments thereof, and many details have been set forth for purpose of illustration, it will be apparent to those skilled in the art that the invention is susceptible to additional embodiments and that certain of the details described herein can be varied considerably without departing from the basic principles of the invention.

What is claimed is:

1. A process for producing an apertured film-coated nonwoven material comprising the steps of:
forming a nonwoven material layer;
extruding a polymer film onto the nonwoven material layer to form a film-coated nonwoven material, the film-coated nonwoven material including a film layer having a thickness not greater than about 0.30 mils; and
forming apertures in at least the film layer to form the apertured film-coated nonwoven material.

2. The process of claim 1 wherein the film layer has a thickness of from about 0.10 mils to about 0.28 mils.

3. The process of claim 1 wherein the nonwoven material layer comprises a spunbond nonwoven web.

4. The process of claim 3 wherein the spunbond nonwoven web comprises a plurality of continuous bicomponent fibers.

5. The process of claim 1 wherein the nonwoven material layer comprises a fiber size gradient structure.

6. The process of claim 1 wherein the nonwoven material layer comprises a bonded carded web including a plurality of discontinuous staple fibers.

7. The process of claim 1 wherein the nonwoven material layer comprises a material selected from a spunbond web, a bonded carded web, a meltblown web, an airlaid material, a coform material, and combinations thereof.

8. The process of claim 1 wherein the nonwoven material layer has a basis weight of about 0.4 osy to about 5.0 osy.

9. The process of claim 1 wherein the nonwoven material layer is treated with a surfactant.

10. The process of claim 1 wherein the polymer film comprises a polymer selected from low density polyethylene, linear low density polyethylene, polypropylene, homopolymers and copolymers, and combinations thereof.

11. The process of claim 1 wherein the polymer film comprises at least one elastomeric polymer.

12. The process of claim 1 wherein the polymer film comprises at least one of a filler and a pigment.

13. The process of claim 1 wherein the nonwoven material layer is treated with a skin wellness additive.

14. The process of claim 1 wherein the polymer film layer comprises a coextruded film having at least a first layer including a polyolefin and a second layer including an adhesive-type polymer.

15. The process of claim 1 wherein the apertures extend at least partially into the nonwoven material layer.

16. The process of claim 1 wherein the apertures extend through the nonwoven material layer.

17. The process of claim 1 wherein the aperturing step includes feeding the film-coated nonwoven material through a nip formed between a pin roll and a corresponding counter roll.

18. The process of claim 17 wherein the pin roll comprises a plurality of pins, each pin extending into the nip about 0.5 mm to about 5.0 mm.

19. The process of claim 17 wherein the pin roll comprises a plurality of pins, each pin having a pin temperature of about 80° C. to about 125° C.

20. The process of claim 17 wherein the film layer faces the pin roll.

21. The process of claim 17 wherein the film layer faces the counter roll.

22. The process of claim 17 wherein the counter roll has a temperature gradient of at least about 5° C.

23. The process of claim 17 wherein the pin roll comprises a plurality of pins, each pin lubricated prior to contacting the film-coated nonwoven material.

24. The process of claim 1 further comprising the step of crimping the nonwoven material layer.

25. The process of claim 1 further comprising the step of applying an adhesive to the nonwoven material layer prior to the film coating step.

26. The process of claim 1 further comprising the step of microembossing the film layer.

27. The process of claim 1 further comprising the step of laminating the apertured film-coated nonwoven material to a material layer.

28. The process of claim 1 wherein the apertured film-coated nonwoven material comprises one of a surge material, a liner, a spacer layer, an extensible ear, a panty liner cover or an outercover.

29. The process of claim 1 wherein the aperturing step includes one of ultrasonic aperturing, hydro entangling aperturing, or passing the film-coated nonwoven material between a pattern calendar and an anvil.

30. A process for producing a film-coated nonwoven material having a plurality of undulations comprising the steps of:
forming a nonwoven material layer;
extruding a polymer film onto a surface of the nonwoven material layer to form a film-coated nonwoven material, the film-coated nonwoven material comprising a film layer having a thickness not greater than about 0.30 mils;
feeding the film-coated nonwoven material through a nip formed between a pin roll and a corresponding counter roll, a film-coated surface of the film-coated nonwoven material facing the pin roll; and
forming a plurality of three-dimensional cones on the film-coated surface.

31. The process of claim 30 wherein an aperture is formed in each of a plurality of valleys formed between adjacent three-dimensional cones.

32. The process of claim 30 wherein the pin roll comprises a plurality of pins, each pin having a conical cross sectional area along a height of the pin.

33. The process of claim 30 wherein the counter roll comprises one of a resilient rubber material, a steel material and a silicone material.

34. The process of claim 30 wherein the film layer has a thickness of from about 0.10 mils to about 0.28 mils.

35. The process of claim 30 further comprising the step of microembossing the film layer.

36. An apertured, film-coated nonwoven material comprising:
a nonwoven material layer having a basis weight of about 0.4 osy to about 5.0 osy;
a polymer film layer extruded onto a surface of the nonwoven material layer, the polymer film layer having a thickness not greater than about 0.28 mils; and
a plurality of apertures formed in at least the polymer film layer.

37. The apertured, film-coated nonwoven material of claim 36 wherein the nonwoven material layer comprises a material selected from a spunbond web, a bonded carded web, a meltblown web, an airlaid material, a coform material, and combinations thereof.

38. The apertured, film-coated nonwoven material of claim 36 wherein the polymer film layer comprises a polymer selected from a low density polyethylene, linear low density polyethylene, polypropylene, homopolymers and copolymers, and combinations thereof.

39. The apertured, film-coated nonwoven material of claim 36 wherein at least one of the nonwoven material layer and the film layer is treated with a surfactant.

40. The apertured, film-coated nonwoven material of claim 36 wherein the polymer film layer comprises at least one elastomeric polymer.

41. The apertured, film-coated nonwoven material of claim 36 wherein the polymer film layer comprises at least one of a filler and a pigment.

42. The apertured, film-coated nonwoven material of claim 36 wherein at least one of the nonwoven material layer and the polymer film layer is treated with a skin wellness additive.

43. The apertured, film-coated nonwoven material of claim 36 wherein the polymer film layer comprises a coextruded film having a first layer including a polyolefin and a second layer including an adhesive-type polymer.

44. The apertured, film-coated nonwoven material of claim 36 wherein the plurality of apertures each extends through the film layer.

45. The apertured, film-coated nonwoven material of claim 36 wherein the plurality of apertures each extends at least partially into the nonwoven material layer.

46. The apertured, film-coated nonwoven material of claim 36 wherein the plurality of apertures each extends through the nonwoven material layer.

47. The apertured, film-coated nonwoven material of claim 36 wherein the nonwoven material layer is pattern-unbonded fabric pattern.

48. An absorbent article comprising:
a liner including a nonwoven material layer, at least one surface of the liner having a pattern-unbonded pattern thereon; a polymer film layer extruded onto the at least one surface of the nonwoven material layer having the pattern-unbonded pattern thereon, the polymer film layer having a thickness not greater than about 0.30 mils; and a plurality of apertures formed through the liner material;
a backsheet joined to the liner; and
an absorbent core disposed and enclosed between the liner and the backsheet.

49. The absorbent article of claim 48 wherein the nonwoven material layer comprises one of a spunbond web, a meltblown web, a bonded carded web, an airlaid web, a coform material or a laminate thereof.

50. The absorbent article of claim 48 wherein the film layer has a thickness not greater than about 0.20 mils.

51. The absorbent article of claim 48 wherein the film layer comprises a co-extruded film layer including a polyolefin polymer layer and an adhesive-type polymer film layer.

52. The absorbent article of claim 48 wherein the film layer comprises at least one polymer selected from the group consisting of polypropylene, low density polyethylene, liner low density polyethylene, a copolymer and combinations thereof.

53. The absorbent article of claim 48 wherein at least one of the film layer and the nonwoven material layer comprises a surfactant.

54. The absorbent article of claim 48 wherein the film layer comprises a skin wellness additive.

55. The absorbent article of claim 48 wherein the film layer comprises at least one of a filler and a pigment.

56. The absorbent article of claim 48 wherein the film layer comprises an elastomeric polymer.
